(19) United States
(12) Patent Application Publication
(10) Pub. No.: US 2026/0204355 A1
(43) Pub. Date: Jul. 16, 2026

(54) METHOD FOR IMPROVED GLYCOPEPTIDE IDENTIFICATION

(71) Applicant: THE TRUSTEES OF INDIANA UNIVERSITY, Bloomington, IN (US)

(72) Inventors: Haixu TANG, Bloomington, IN (US); Sujun LI, Zionsville, IN (US)

(21) Appl. No.: 19/132,995

(22) PCT Filed: Nov. 28, 2023
(86) PCT No.: PCT/US2023/081309

§ 371 Date: May 27, 2025

Related U.S. Application Data
(60) Provisional application No. 63/428,311, filed on Nov. 28, 2022.

(51) Int. Cl. G16B 40/10; G16B 35/10; G16B 35/20

(52) U.S. Cl. CPC G16B 40/10; G16B 35/10; G16B 35/20

(57) ABSTRACT

Liquid chromatography coupled with tandem mass spectrometry (LC-MS/MS) is commonly adopted in large-scale glycoproteomic studies involving hundreds of disease and control samples. Current methods for glycopeptide identification in such data analyze the individual datasets and do not exploit redundant spectra of glycopeptides present in related datasets. A concurrent approach is provided for glycopeptide identification in multiple related glycoproteomic datasets by using spectral clustering and spectral library searching.

### CROSS-REFERENCE TO RELATED APPLICATION

This application claims priority to, and incorporates by reference, U.S. provisional application with Ser. No. 63/428,311 filed on Nov. 28, 2022.

### STATEMENT OF FEDERALLY SPONSORED RESEARCH

This invention was made with government support under CA225753 and GM130091 awarded by the National Institutes of Health. The government has certain rights in the invention.

### BACKGROUND

The last decade has witnessed the rapid advancement of mass spectrometric techniques, in particular, liquid chromatography coupled tandem mass spectrometry (LC-MS/MS), which provides drastically improved sensitivity and throughput. As a result, LC-MS/MS has become the dominant technique for not only identifying and quantifying proteins in complex proteome samples, but also characterizing post-translational modifications (PTMs) in proteins, including site-specific protein glycosylations, by using glycoproteomic approaches. To automate glycoproteomic data analyses, many software tools, such as pGlyco, Byonic, I-GPA, GP Finder, gFinder, GlycoMaster DB, GlycoPep grader, GlycoPep Detector, and GlycoPeptideSearch (GPS) have been developed for the characterization of glycopeptides from the MS/MS spectra.

Despite the progress, several challenges still remain in glycopeptide identification, which largely impede the comparative analyses of large-scale glycoproteomic data acquired from large cohorts of patients with complex diseases such as cancer. First, existing algorithms employ ad hoc scoring schemes derived empirically from a small number of annotated MS/MS spectra of glycans and glycopeptides; however, they may not be well generalized to spectra of other glycopeptides. Second, the methods for estimating false identification rate (FDR) in glycopeptide identifications have not been systematically validated, which may sometimes over-estimate the FDR in the identification results. Finally, large-scale human glycoproteomic experiments can generate datasets from tens to hundreds of individual disease or control samples (e.g., in an attempt to identify quantitative biomarkers), often containing many spectra from identical glycans/glycopeptides at different abundances across these samples. However, there is a lack of algorithms that can exploit the redundant information in these cohort-based and related studies to improve and speed up glycopeptide identification.

### SUMMARY OF THE DISCLOSURE

The present disclosure addresses the aforementioned drawbacks associated with the identification of glycopeptides from their tandem mass spectra (MS/MS) in large scale glycoproteomic analyses using LC-MS/MS by providing methods and system for spectral clustering and spectral library searching.

In one aspect of the disclosure, a method for identifying glycopeptides in mass spectrometry datasets is provided. The method includes accessing a plurality of mass spectrometry datasets, where each of the plurality of mass spectrometry datasets comprises mass spectrometry spectra acquired from a different sample; generating spectral cluster data comprising a plurality of spectral clusters by clustering the plurality mass spectrometry spectra within each of the plurality of mass spectrometry datasets; generating a consensus spectrum for each spectral cluster in the spectral cluster data; performing a comparison of each of the consensus spectra to a glycopeptide database containing a plurality of spectra of a plurality of known glycopeptides; identifying the plurality of glycopeptides in each of the plurality of consensus spectra based on the comparison; generating a plurality of labeled consensus spectra based on the plurality of glycopeptides identified in each of the plurality of consensus spectra; comparing the plurality of mass spectrometry spectra in the plurality of mass spectrometry datasets with the plurality of labeled consensus spectra within each of the plurality of related datasets; and identifying the plurality of glycopeptides in the mass spectrometry spectra in the plurality of mass spectrometry datasets based on comparing the plurality of labeled consensus spectra with the mass spectrometry spectra in the plurality of mass spectrometry datasets.

In another aspect of the disclosure, a method for identifying glycopeptides in mass spectrometry datasets may include accessing a first plurality of mass spectrometry datasets, wherein each of the plurality of mass spectrometry datasets comprises mass spectrometry spectra acquired from a first multiplicity of samples; generating spectral cluster data comprising a plurality of spectral clusters by clustering the plurality mass spectrometry spectra within each of the first plurality of mass spectrometry datasets; generating a consensus spectrum for each spectral cluster in the spectral cluster data; performing a comparison of each of the consensus spectra to a glycopeptide database containing a plurality of spectra of a plurality of known glycopeptides; identifying the plurality of glycopeptides in each of the plurality of consensus spectra based on the comparison; generating a plurality of labeled consensus spectra based on the plurality of glycopeptides identified in each of the plurality of consensus spectra; comparing a second plurality of mass spectrometry spectra in a second plurality of mass spectrometry datasets generated from a second multiplicity of samples with the plurality of labeled consensus spectra; and identifying the plurality of glycopeptides in the second multiplicity of samples based on comparing the plurality of labeled consensus spectra acquired from a first multiplicity of samples, with the mass spectrometry spectra in the second plurality of mass spectrometry datasets, wherein the first multiplicity of samples and the second multiplicity of samples are prepared using the same experimental protocol.

In one aspect of the disclosure, a system for identifying glycopeptides in multiple related datasets is described, the system comprising: a processor configured to: acquire a plurality of liquid chromatography coupled tandem mass spectrometry (LC-MS/MS) spectra from a plurality of related datasets; cluster the plurality of LC-MS/MS spectra within each of the plurality of related datasets; generate a consensus spectrum for each of the LC-MS/MS spectra within each of the plurality of related datasets; perform a comparison of each of the consensus spectra to a glycopeptide database containing a plurality of spectra of a plurality of known glycopeptides; identify the plurality of glycopeptides in each of the plurality of consensus spectra based on the comparison; generate a plurality of labeled consensus spectra based on the plurality of glycopeptides identified in each of the plurality of consensus spectra; compare the plurality of LC-MS/MS spectra with the plurality of labeled consensus spectra within each of the plurality of related datasets; and identify the plurality of glycopeptides in the plurality of LC-MS/MS spectra based on the plurality of labeled consensus spectra.

### BRIEF DESCRIPTION OF THE DRAWINGS

FIG. 1 illustrates an example workflow for concurrent glycopeptide identification by using spectral library searching (GlycoSLASH) according to an aspect of the present disclosure.

FIG. 2 is a flowchart describing the steps of the method according to an aspect of the present disclosure.

FIG. 3 is an example consensus spectrum compared to an individual spectrum according to an aspect of the present disclosure.

FIG. 4 shows the purity of spectral clusters with different similarity cutoff for each charge, according to an aspect of the present disclosure.

FIG. 5 shows the abundances (measured by the total spectral counts) of the most significant glycoproteomic features in Dataset I (top) and II (bottom), according to an aspect of the present disclosure.

FIG. 6 shows a diagram of an example computer system for implementation of the methods according to aspects of the present disclosure.

FIG. 7 shows a block diagram of example components that can implement the system of FIG. 6.

### DETAILED DESCRIPTION

The present disclosure presents a novel concurrent approach, named GlycoSLASH, for glycopeptide identification in multiple related glycoproteomic datasets by using spectral clustering and spectral library searching, which leverages the redundant glycopeptides shared in related samples (e.g., in a disease/control cohort studies) to improve the identification results. The approach generally includes the following steps: 1) prior to identification, all MS/MS spectra from multiple datasets (e.g., each from the LC-MS/MS analysis of the serum sample from a disease patient or a control subject) are clustered using a spectral clustering algorithm, such as msCRUSH; 2) a spectral library containing the consensus spectra, each from one spectral cluster, is constructed; 3) identification within each cluster is mapped to locate the consensus identification; and 4) the MS/MS spectra from the whole dataset are searched against the spectral library using a library searching algorithm, such as msSLASH, to identify the glycopeptides based on the constructed glycopeptides library.

In an example study, the disclosed approach was evaluated on two large-scale glycoproteomic studies: one involving 533,248 spectra, and the other involving 1,561,882 spectra. The results showed that the concurrent approach can identify 105%-224% more spectra as glycopeptides compared to the glycopeptide identification on individual datasets by using existing analysis techniques, such as Byonic, alone. Furthermore, based on the improved glycopeptide identification results, the label-free quantification of site-specific or glycan type-specific glycosylations can be achieved by using the spectral counting approach that was commonly adopted in proteomics. The results suggested several potential glycoproteomic biomarkers to distinguish hepatocellular carcinoma and cirrhosis patients.

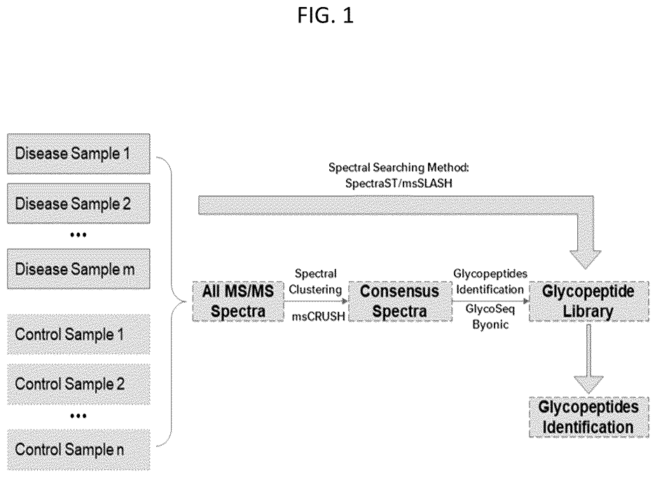

FIG. 1 illustrates an example workflow of GlycoSLASH for concurrent glycopeptide identification from multiple datasets acquired from relevant glycoproteomic samples. First, all MS/MS spectra from all input datasets are clustered using a spectral clustering algorithm, such as the msCRUSH algorithm, and for each cluster a consensus spectrum is generated. If a cluster contains one or more spectra identified as a glycopeptide by a glycopeptide identification algorithm (e.g., Byonic or GlycoSeq), it is annotated as the most frequently identified glycopeptide. For example, if a cluster contains 15 spectra, in which 10 were identified as the glycopeptide A, two were identified as the glycopeptide B and the remaining three were not identified as glycopeptides, the cluster (and its consensus spectrum) is annotated as the glycopeptide A. In the two large-scale example glycoproteomic studies conducted to assess the disclosed methods, most (more than 90.9%) clusters of spectra either identified as the same glycopeptide or were not identified. As a non-limiting example, the consensus spectra can be generated using the empirical procedure implemented in msCRUSH.

Spectral clustering reduces the false identification of glycopeptides (i.e., similar MS/MS spectra that were identified as different glycopeptides), and thus produces consistent results for concurrent glycopeptide identification across multiple samples. In addition to the annotation of glycopeptides, the spectral clusters were annotated as an unmodified peptide if some spectra in the cluster were identified as one or more unmodified peptides by a database searching algorithm (e.g., Mascot or MSGF+). As an example, the annotation rule implemented in this step can be the same as described above for glycopeptides. The consensus spectra from the clusters annotated as glycopeptides or peptides are then assembled into a spectral library. Finally, the glycopeptide identification is performed by searching the MS/MS spectra in all input spectra against the spectral library using a spectral searching algorithm, such as msSLASH. As a non-limiting example, the spectra sharing a cosine similarity, greater than a threshold value, with a consensus spectrum in the spectral library annotated as a glycopeptide is then identified as the annotated glycopeptide. Here, the false discovery rate (FDR) of the spectral library searching results with a similarity threshold (e.g., 0.6 by default) can be estimated using results of peptide identification performed simultaneously: if an MS/MS spectrum is identified as an unmodified peptide by the database searching algorithm, but shares the similarity greater than the threshold with a consensus spectrum annotated as a glycopeptide, itis considered as a false identification.

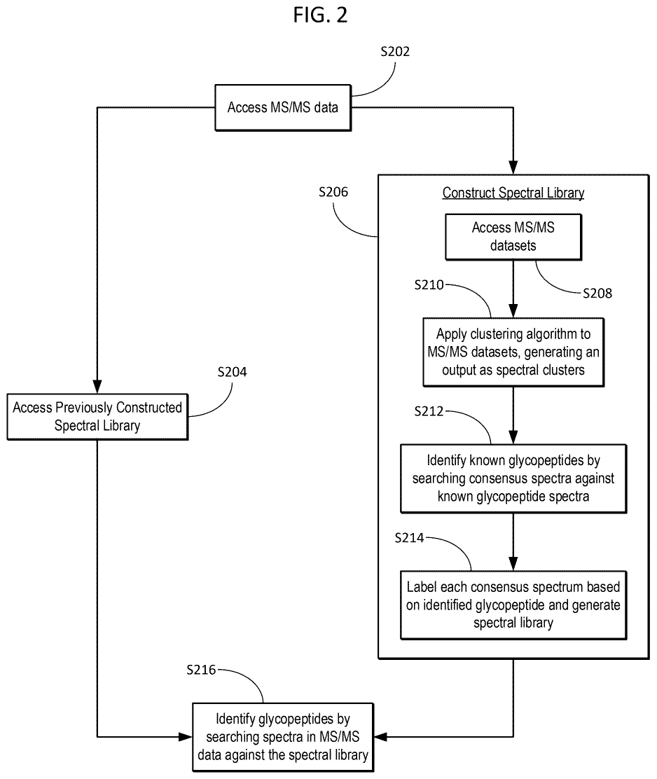

FIG. 2 illustrates the steps of an example method for identifying peptides in a sample by performing the GlycoSLASH workflow. The method includes accessing tandem mass spectrometry (i.e., MS/MS) data with a computer system, as indicated at step S202. Accessing the MS/MS data may include retrieving such data from a memory, database, or other suitable data storage device or medium. Additionally or alternatively, accessing the MS/MS data may include acquiring such data with a mass spectrometry system and transferring or otherwise communicating the data to the computer system, which may be a part of the mass spectrometry system.

The method also includes accessing a spectral library with the computer system, as indicated at step S204. Accessing the spectral library may include retrieving such a library from a memory, database, or other suitable data storage device or medium. Additionally or alternatively, accessing the spectral library may include constructing the spectral library from mass spectrometry data sets, as indicated at step S206. For instance, constructing the spectral library can include accessing or otherwise acquiring a plurality of MS/MS data sets, generating spectral clusters by performing spectral clustering on the MS/MS data sets, determining consensus spectra from the spectral clusters, and constructing the spectral library based on the consensus spectra, as described above.

As a non-limiting example of constructing a spectral library, MS/MS spectra are first acquired, or previously acquired spectra are accessed, for samples from related datasets, as indicated at step S208. In one example, two datasets were used to evaluate the performance of GlycoSLASH. In some cases, a first plurality of mass spectrometry datasets may be acquired from a first multiplicity of samples and a second plurality of mass spectrometry datasets may be acquired from a second multiplicity of samples. The first plurality of mass spectrometry datasets may be used to construct a spectral library while, for example, the second plurality of mass spectrometry datasets may be used to query the spectral library in order to identify a plurality of glycopeptides in the second plurality of mass spectrometry datasets. In some cases, the origin of, and/or experimental protocols for acquiring, the first multiplicity of samples and second multiplicity of samples may be the same. For example, the first multiplicity of samples and second multiplicity of samples may both originate as blood serum (i.e., human blood serum). In a further example, the first multiplicity of samples and second multiplicity of samples may both be immunopurified, digested, and/or enriched. In some cases, the first multiplicity of samples and second multiplicity of samples may be the same samples and from the same dataset (i.e., the first multiplicity of samples is Dataset I, further described below, and the second multiplicity of samples is Dataset I). In some cases, the first multiplicity of samples and second multiplicity of samples may be different samples and form the different datasets (i.e., the first multiplicity of samples is Dataset I and the second multiplicity of samples is Dataset II, further described below).

Dataset I (ProteomeXchange ID PXD011239) studied the site-specific N-glycosylations in haptoglobin in the blood samples from the patients of hepatitis C virus (HCV)-related liver cirrhosis and early hepatocellular carcinoma (HCC). Specifically, haptoglobin was immunopurified from the serum of patients with early stage HCC, liver cirrhosis, and healthy controls, followed by trypsin and GluC digestion, glycopeptide enrichment and LC-EThcD-MS/MS analysis on an Orbitrap Fusion Lumos Tribrid mass spectrometer (Thermo). Identification and differential quantitation of site-specific N-glycopeptides were performed using a combination of Byonic and Byologic software. This dataset included 39 raw mass spectrometry files acquired from 5 early stage HCC, 5 liver cirrhosis patients, and 5 healthy controls, with a total of 533,248 MS/MS spectra. The glycopeptides on the N-glycosylation sites of Asn184 (N184), Asn207 (N207), Asn241 (N241) and Asn211 (N211) were identified.

Dataset II (ProteomeXchange via ID PXD01850236) exploited the same experimental protocol to study the site-specific N-glycosylations in haptoglobin from the patients of liver cirrhosis, early, and late stage HCC with nonalcoholic steatohepatitis (NASH). This dataset included 140 LC-EThcD-MS/MS raw mass spectrometry files acquired from 22 early stage HCC, 15 late stage HCC, and 33 liver cirrhosis patients, with a total of 1,561,882 MS/MS spectra.

At step S210, the MS/MS spectra are clustered within each dataset. In an example, msCRUSH was employed to cluster the spectra from the aforementioned dataset I. The parameters for msCRUSH are set as the following: 1) number of hash functions per hash table: 15; 2) number of iterations: 200; 3) clustering threshold: 0.6, 4) minimum and maximum m/z for spectra representation: 200 and 2000. Consensus spectra are then determined for each cluster, as described above.

The consensus spectra are then compared to spectra of known glycopeptides to identify the glycopeptide of each consensus spectrum, as indicated at step S212. In an example, the commercial software Byonic was employed for the initial glycopeptides identification. The search was performed using the following parameters: (1) static modification: carbamidomethyl of C; (2) dynamic modifications: oxidation of M, deamindation of N and Q and glycan modifications; (3) maximum miss cleavages: 2; (4) precursor mass tolerance: 10 ppm; (5) MS/MS mass tolerance: 0.01 Da; (6) 1% FDR filter and threshold of Byonic score is higher or equal than 100. MASCOT was also performed on both datasets to identify unmodified peptides that remained in the sample after the experimental procedure for haptoglobin enrichment. The parameters for MASCOT were set as the following: 1) carbamidomethyl as fixed modification on Cystine; 2) oxidation as variable modification on methionine; 3) peptide mass tolerance: 0.5 Da; 4) MS/MS mass tolerance: 0.2 Da; and 5) allowing up to 1 missed cleavage.

In step S214 labeled consensus spectra are generated based on the chosen glycopeptide identified for each consensus spectra in the previous step. The labeled consensus spectra form the spectral library. For the library construction, the Byonic software identification and MASCOT peptide identification were first mapped to each spectrum. After the spectral clustering, the most abundant common identification among each cluster was chosen to represent the cluster. For example, if one cluster includes 6 spectra, 5 out of these 6 spectra are all identified as peptide A, then the consensus spectrum will be labeled as peptide A in the spectral library. For any singleton clusters that are formed during the spectral clustering, each singleton cluster is represented by the only spectrum it contains. The clustering and spectral library construction can be performed for each charge state. In the example study, for charge 2+, 606 consensus spectra were constructed, among which 68 glycopeptides were represented. For charge 3+, 387 consensus spectra with 188 glycopeptides; for charge 4+, 195 in total with 176 glycopeptides and for charge 5+, 27 in total with 22 glycopeptides, were constructed, respectively.

Next, the MS/MS spectra acquired or otherwise accessed in step S202 are searched against the spectral library in S216. As a non-limiting example, msSLASH can be used for spectral library searching, in which the locality-sensitive hashing (LSH) technique is employed to increase the search speed. The threshold of cosine similarity between the input and consensus spectrum in the library was set as 0.6, by may be selected from a range of 0.6-0.8. inclusive; the consensus spectrum with the similarity higher than the threshold was used to identify the input MS/MS spectrum. The FDR corresponding to the similarity threshold estimated by using unmodified peptide spectra is below 0.01.

### Example 1

### Spectral Clustering and Library Construction

To evaluate the performance of the GlycoSLASH method, a glycopeptide spectral library was first built using the MS/MS spectra from Dataset I, and then glycopeptides identified for both Dataset I and II by spectra searching against the library. Because Dataset I and II were acquired from human serum using the same experimental protocol, the spectral library built from one dataset was tested to determine whether it is sufficient for glycopeptide identification from related datasets.

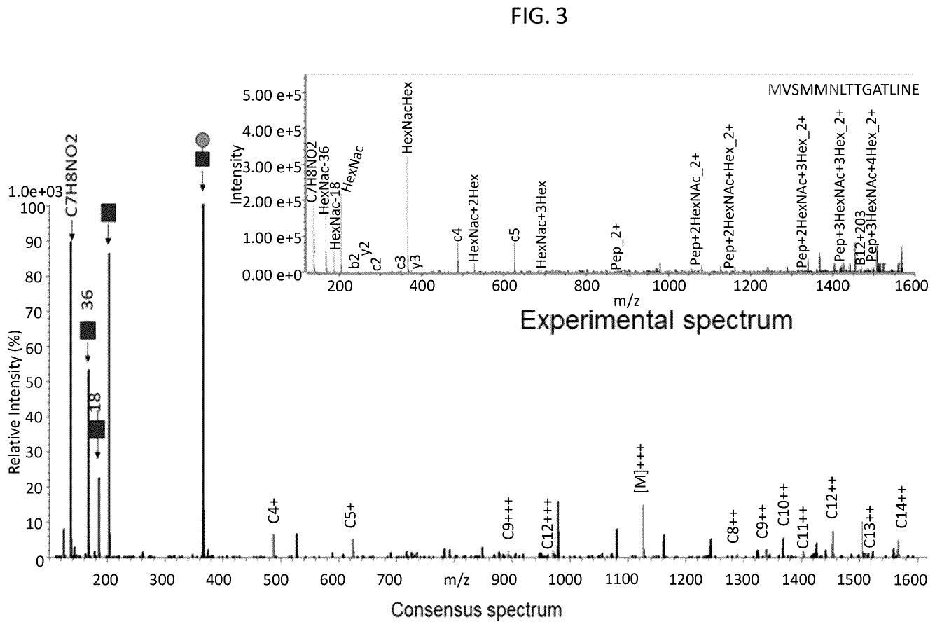

Dataset I contain 39 raw LC-MS/MS files and 533,248 MS/MS spectra in total. msCRUSH38 was employed to cluster these spectra. 312,479 spectra in charge 2+ were clustered into 136,926 clusters (43.8% reduction), 119,127 spectra in charge 3+ were clustered into 70,616 clusters (59.3% reduction), 81,419 spectra in charge 4+ were clustered into 56,865 clusters (69.8% reduction), and 17,943 spectra in charge 5+ were clustered into 12,501 clusters (69.7% reduction). Among these clusters, 86.6% clusters in 2+ are singletons (containing only one spectrum in the cluster); similarly, 88.9% 3+, 92.6% 4+ and 93.2% 5+ spectra are singletons, respectively. In addition, 6.1% 2+ clusters, 5.4% 3+ clusters, 3.5% 4+ clusters and 3.2% 5+ clusters contain two spectra, and the remaining less than 5% clusters contain more than two spectra.

| A spectral library was constructed using the consensus spectra representing the clusters of spectra with the charges +2 to +5 that are annotated as unmodified peptides or glycopeptides. Notably, several redundant clusters may share the same annotation (e.g., the same glycopeptides). In these cases, only the consensus spectrum of the largest clusters in the library (e.g., for two clusters annotated as the same glycopeptides with three and one spectra, respectively, the consensus spectra of the cluster with three spectra in the library was incorporated) were retained. Finally, the library includes 1,215 spectra representing the clusters of the charges 2+ to 5+ (Table 1), including 454 (37.4%) annotated as the glycopeptides. For examples, among the 136,927 clusters of the 2+ spectra, 1,607 were annotated as unmodified peptides or glycopeptides. After eliminating redundant clusters, 606 consensus spectra were incorporated in the library, including 68 (11.2%) annotated as glycopeptides, and the remaining 538 annotated as unmodified peptides. In comparison, among the 70,596 clusters of the 3+ spectra, 387 consensus spectra were incorporated in the library, including 188 (48.5%) annotated as glycopeptides. FIG. 3 shows a spectrum annotation example as consensus spectrum and an individual spectrum.

TABLE 1
Spectral library constructed from the clustering of Dataset I.

| | Charge 2+ | Charge 3+ | Charge 4+ | Charge 5+ | Total |
|---|---|---|---|---|---|
| Spectra | 312,479 | 119,127 | 81,419 | 17,943 | 533,248 |
| Identified Spectra | 7,268 | 3,034 | 2,278 | 72 | 12,652 |
| Clusters | 136,927 | 70,596 | 56,866 | 12,502 | 276,891 |
| Annotated Clusters | 1,001 | 962 | 1,053 | 45 | 3,061 |
| Spectra in the Library | 606 | 387 | 195 | 27 | 1,215 |
| Glycopeptide Spectra | 68 | 188 | 176 | 22 | 454 |
| Percentage | 11.2% | 48.5% | 90.2% | 81.5% | 37.4% |

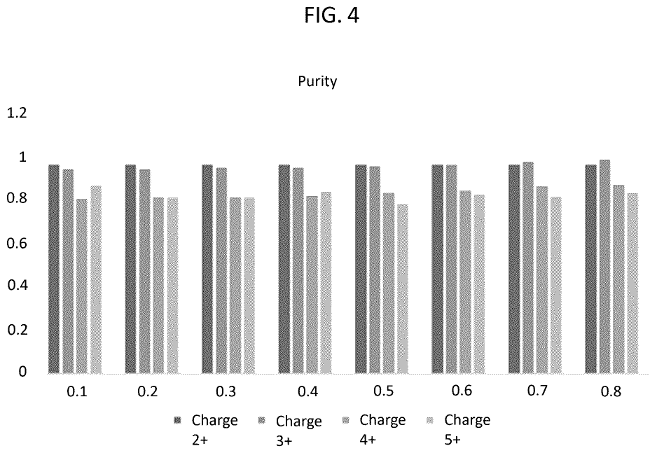

Furthermore, the purity of the spectral clusters was examined using the identification results of both MASCOT and Byonic. Here, the purity is calculated by the formula depicted in the reference msCRUSH, where the spectra in a cluster identified as a glycopeptide by Byonic or as an unmodified peptide by MASCOT are considered to be different. FIG. 4 shows the purity values for each spectral cluster with different similarity cutoff. For example, the clusters with similarity cutoff of 0.6 (which is the default cutoff for peptide spectral clustering) are 0.97 for charge 3+ spectra and 0.85 for charge 4 spectra, respectively, which remain almost the same when the similarity cutoff is increased to 0.9. To be noted here, only the clusters with multiple identified spectra were considered in the purity calculation. Overall, these results suggest the spectral clustering reported by msSLASH with the default cutoff 0.6 are accurate and consistent.

### Glycopeptiede Identification by Spectral Library Searching

The constructed spectral library (containing 454 consensus spectra annotated as glycopeptides) is subject to spectral library searching for concurrent glycopeptide identification for each sample in dataset I and II, respectively. The cutoff of cosine similarity was selected as 0.6; the query MS/MS spectrum is identified based on the annotation of the spectrum in the library with the highest similarity if it is greater than the cutoff.

As shown below, the estimated false discovery rate (FDR) using the similarity cutoff 0.6 is much lower than 0.01. Table 2 shows the identification of MS/MS spectra of different charges in dataset I. GlycoSLASH identified a total of 3,431 and 3,085 MS/MS spectra as glycopeptides in the charge 3+ and charge 4+, respectively. In comparison, Byonic identified 1,605 3+ spectra as glycopeptides, in which 1,517 spectra were identified as the identical glycopeptides as those identified by GlycoSLASH, while 65 (4.05%) were identified as different glycopeptides. GlycoSLASH identified 1,827 more spectra as glycopeptides that were not identified by Byonic. Similarly, Byonic identified 1,656 spectra of the charge 4+ as glycopeptides, in which 239 (14.43%) were identified as different glycopeptides from those identified by GlycoSLASH.

TABLE 2
Peptide and glycopeptide identification by GlycoSLASH on Dataset I.

| | charge 2+ | charge 3+ | charge 4+ | charge 5+ | Total |
|---|---|---|---|---|---|
| Identification | 14,618 | 6,344 | 3,582 | 268 | 24,812 |
| Glycopeptides ID | 413 | 3,431 | 3,085 | 78 | 7,007 |
| Peptide ID | 14,205 | 2,913 | 497 | 190 | 17,805 |
| Byonic | 248 | 1,605 | 1,656 | 53 | 3,562 |
| Byonic(MisMatched) | 0 | 65 | 143 | 1 | 209 |
| MASCOT | 6,862 | 1,148 | 178 | 14 | 15,064 |
| MASCOT (MisMatched) | 32 | 0 | 0 | 0 | 32 |
| GlycoSLASH New ID | 7,508 | 3,595 | 1,748 | 201 | 13,052 |
| New ID(Glycopeptides) | 165 | 1,827 | 1,429 | 25 | 3,446 |

Estimation of the FDR of the spectral library searching was performed by comparing the identification results of unmodified peptides by GlycoSLASH (using the same similarity cutoff of 0.6) with those by MASCOT. For the spectra in charge 2+, MASCOT identified 6,862 spectra as unmodified peptides, in which only 32 (0.05%) are different from the identification of GlycoSLASH that reported the library spectra annotated as different unmodified peptides or a glycopeptides. For spectra with the charges 3+ and 4+, all unmodified peptides identified by MASCOT are identical to those by GlycoSLASH. Based on these results, it can be concluded that if the similarity between the reported library spectrum and the query spectrum is greater than the cutoff (0.6), much fewer than 1% of these pairs are from different peptides, considering the MASCOT identification results are all correct. Therefore, it was estimated that the FDR of the spectral library search at the cutoff 0.6 is much lower than 1%.

The characteristics of these spectra with mismatched glycopeptide identification between Byonic and GlycoSLASH were further investigated. The complete set of these spectra along with their identifications (including 65 3+, 143 4+ and one 5+ spectra) are shown in the supplementary table 1. The cosine similarity between each of these spectra and the other spectra with the same glycopeptide identification was calculated. The 65 3+ spectra were identified by the spectral library searching method as the same glycopeptides as 3,837 other spectra. The average cosine similarity between these spectra pairs identified by GlycoSLASH is about 0.77. In comparison, these spectra were identified as the same glycopeptides by Byonic as 2,497 other spectra, and the average cosine similarity between these spectra pairs is 0.74. Similarly, the 143 4+ spectra were identified as the same glycopeptides as 5,513 and 2,985 other spectra by GlycoSLASH and Byonic, respectively. The average cosine similarity between the spectrum pairs is 0.70 for GlycoSLASH, and 0.66 for Byonic. These results suggest that among the spectra identified as different glycopeptides by GlycoSLASH and Byonic, in general it is more likely that the GlycoSLASH identification results are correct. On the other hand, the spectral library searching method identified 3,446 (96.7%) additional glycopeptide spectra that were not identified by Byonic, which enables the use of spectral counts as a straightforward glycopeptide quantification method (see below).

Next, the glycopeptides in dataset II were identified by searching the MS/MS spectra in the dataset against the spectral library constructed from dataset I. As described above, these two datasets were acquired by using the same experimental protocol on human serum samples from related patients. Herein, it is demonstrated that the spectral library constructed from one glycoproteomic dataset can be applied to related datasets.

The glycopeptide identification results by GlycoSLASH are shown in Table 3. Dataset II contains 1,561,882 spectra. It is noted here that 5,296 spectra with no charge or >5+ charges were excluded in the analyses (Those spectra are not included in Table 3). Among them, 72,784 were identified by the spectral library searching, in which 32.3% were identified as glycopeptides (including 58.7% 3+ and 91.8% 4+ spectra), leading to the identification of 270 unique glycopeptides. These results and identification ratio are comparable with the results from Dataset I (Table 2).

TABLE 3
Peptide and glycopeptide identification by GlycoSLASH on Dataset II.

| | charge 2+ | charge 3+ | charge 4+ | charge 5+ | Total |
|---|---|---|---|---|---|
| Spectra | 783,488 | 508,764 | 224,766 | 39,568 | 1,561,882 * |
| GlycoSLASH ID | 41,150 | 19,444 | 11,789 | 401 | 72,784 |
| Glycopeptide ID | 1,205 | 11,420 | 10,818 | 66 | 23,509 |
| Peptide ID | 39,945 | 8,024 | 971 | 35 | 49,275 |
| Glycopeptide Percentage | 2.9% | 58.7% | 91.8% | 16.5% | 32.3% |
| Unique Glycopeptides | 18 | 131 | 118 | 5 | 270 |

* 5,296 spectra with no charge or >5+ charges were excluded in the analyses.

### Quantitative Glycopeptide Features Associated with HCC and Cirrhosis

Numerous studies have found that fucosylated/sialylated glycan structures in serum Hp were significantly elevated in HCC patients compared to those in cirrhosis patients. Specifically, previous researches reported that site-specific N-glycopeptides on the N-glycosylation sites N184 and N241 were significantly elevated in early stage HCC patients compared to cirrhosis patients and normal controls using the same datasets when Byonic is employed for glycopeptide quantification.

Herein, the glycopeptides were quantified using a straightforward and robust spectral counting method to investigate potential site-specific glycoproteomic biomarkers. In total, 66 glycoproteomic features were identified. Table 4 shows the features that are significantly associated with the liver diseases: cirrhosis, early or late stage hepatocellular carcinoma (HCC), in the analyses.

TABLE 4
Glycoproteomic features quantified in Dataset I/II

| Feature | Dataset | Description | Pvalue |
|---|---|---|---|
| NFPep_N211 | Dataset I | all unfucosylated N-glycopeptides on N211 | 0.0024 |
| Fuc(1)_bi | Dataset II | all biantennary monofucosylated N-glycoeptides | 0.0006 |
| FucPep_bi | Dataset II | all biantennary fucosylated N-glycoeptides | 0.0008 |
| FucPep | Dataset II | all fucosylated N-glycoeptides | 0.0026 |
| FucPep_N207 | Dataset II | all fucosylated N-glycoeptides on N207 | 0.0054 |
| FucPep_N241 | Dataset II | all fucosylated N-glycoeptides on N241 | 0.0065 |
| Fuc(1)_tri | Dataset II | all monofucosylated triiantennary N-glycoeptides | 0.0097 |

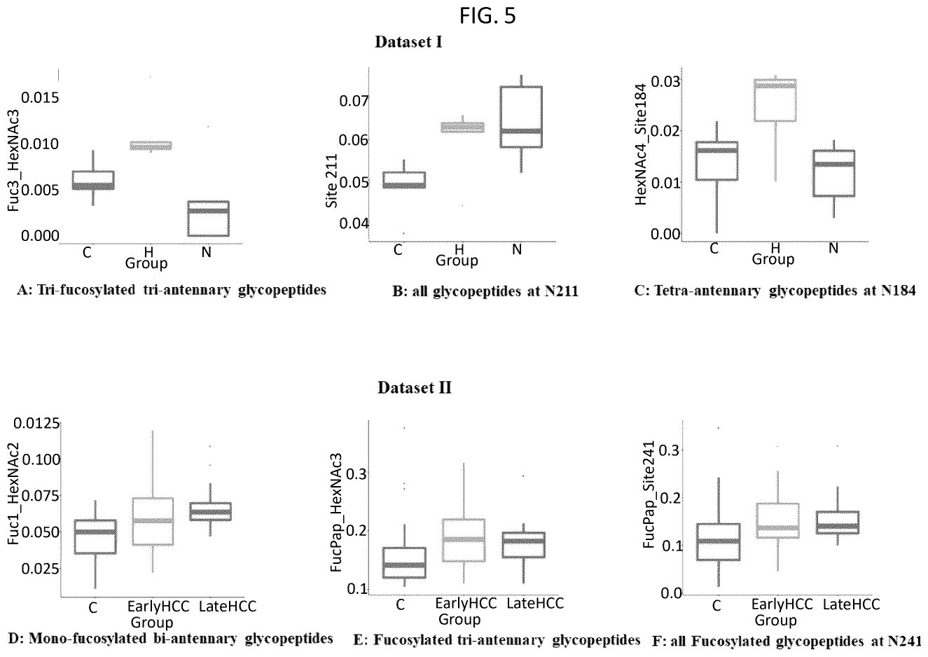

FIG. 5 illustrates the distributions of the abundances in boxplot measured by the total spectral counts of the most significant glycoproteomic features in different types of disease samples: 5 normal, 5 cirrhosis and 5 early stage HCC in Dataset I, and 33 cirrhosis, 22 early and 15 late stage HCC in dataset II. In dataset I, the total abundance of triantennary trifucosylated N-glycopeptides is significantly different in HCC and cirrhosis patients and control samples, which is consistent with previous studies. Interestingly, the total abundances of all glycopeptides on the site N211 of Hp (i.e., the occupancy of the site) is shown to be significantly lower in the HCC patients than in the cirrhosis patients or the control individuals, while the site occupancy is indistinguishable between the cirrhosis patients and the control individuals. On the other hand, the total abundance of tetraantennary N-glycopeptides on the site N184 is significantly higher in the cirrhosis patients than in the HCC patients or the control individuals, while the abundance is indistinguishable between the HCC patients and the control individuals. Similarly, the three features that are distinguishable between the early and late stage HCC and the control individuals are the total abundance of the biantennary monofucosylated N-glycopeptides, the total abundance of all triantennary fucosylated N-glycopeptides and the total abundance of all fucosylated glycopeptides on the site N241. Note that because dataset II includes more samples (22 early stage HCC, 15 late stage HCC and 33 liver cirrhosis) than Dataset I, the quantitative features tend to be less distinguishable among different disease groups.

### Computer Systems

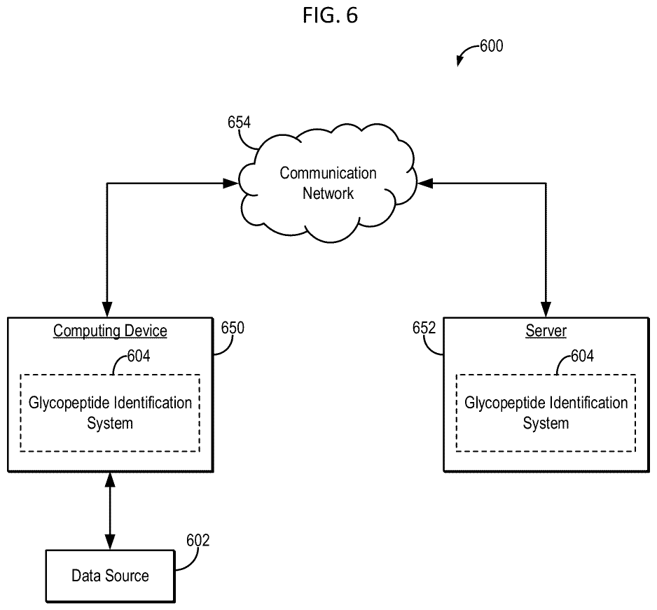

Referring now to FIG. 6, an example of a system 600 for performing glycopeptide identification in accordance with some embodiments of the systems and methods described in the present disclosure is shown. As shown in FIG. 6, a computing device 650 can receive one or more types of data (e.g., MS/MS spectra acquired from an LC-MS/MS system or other tandem mass spectrometry system, previously generated consensus spectra, previously constructed spectral libraries, and so on) from data source 602. In some embodiments, computing device 650 can execute at least a portion of a glycopeptide identification system 604 to identify glycopeptides in MS/MS data acquired from a sample using data received from the data source 602.

Additionally or alternatively, in some embodiments, the computing device 650 can communicate information about data received from the data source 602 to a server 652 over a communication network 654, which can execute at least a portion of the glycopeptide identification system 604. In such embodiments, the server 652 can return information to the computing device 650 (and/or any other suitable computing device) indicative of an output of the glycopeptide identification system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 602 can be any suitable source of data (e.g., MS/MS data, spectral cluster data, consensus spectra, spectral libraries), such as a mass spectrometry system, another computing device (e.g., a server storing MS/MS data, spectral cluster data, consensus spectra, spectral libraries), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

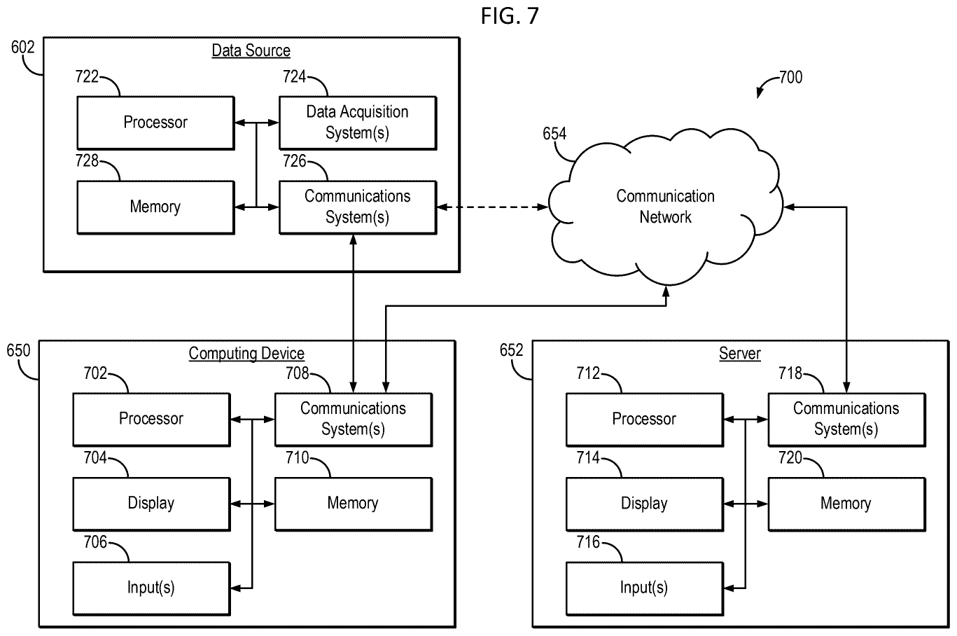

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., spectra, images, user interfaces, graphics, tables), receive content from server 652, transmit information to server 652, and so on. For example, the processor 702 and the memory 710 can be configured to perform the methods described herein (e.g., the workflow of FIG. 1, the method of FIG. 2).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, spectra, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the workflow of FIG. 1, the method of FIG. 2).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire mass spectrometry data, such as MS/MS data, and can include a mass spectrometry system. Additionally or alternatively, in some embodiments, the one or more data acquisition systems 724 can include any suitable hardware, firmware, and/or software for coupling to and/or controlling operations of a mass spectrometry system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate spectra from data; present content (e.g., data, spectra, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate spectra, transmit information and/or content (e.g., data, spectra, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

In the present disclosure, a concurrent approach (named GlycoSLASH) to improve glycopeptide identification by leveraging multiple glycoproteomic datasets from related samples is presented. This approach uses spectral library searching against a glycopeptide spectral libraryconstructed from experimental glycoproteomic data. msSLASH was evaluated on two glycoproteomic datasets acquired from HCC and cirrhosis patients.

Furthermore, the disclosure presents two major techniques named “msCRUSH” and “msSLASH”. While msCRUSH is a rapid algorithm for tandem mass spectral clustering with high sensitivity and accuracy by exploiting the locality-sensitivity hashing (LSH) to speed up the similarity comparison between pairs of mass spectra, msSLASH also employs LSH demonstrating it can significantly improve the efficiency for spectral library searching while retaining the competitive sensitivity. In addition, to generate the consensus spectra, all MS/MS spectra is first clustered from multiple datasets using the spectral clustering algorithm msCRUSH. The consensus spectrum is then computed by merging the peaks in all spectra in each cluster: the peaks in all spectra within certain mass precision are merged into a single peak in the consensus spectrum, for which the m/z is set as the average m/z of all merged peaks weighted by the intensity of each peak, and the intensity is set to be the average intensity of all merged peaks.

Regarding the quantification, peak areas in the extracted ion chromatography (XIC) was commonly used for quantification of specific ion species in MS data, and was also employed in the original studies for the quantification of individual glycopeptides and the identification of potential biomarkers of specific glycopeptides (with the same N-glycans attached to the same peptide backbones). In contrast in GlycoSLASH, the consensus spectra was generated and the spectral similarity was used to perform the spectra searching, which resulted in a more comprehensive glycopeptide identification. This approach enables quantification of not only specific glycopeptides, but also groups of glycopeptides, e.g., the N-glycans attached to the same glycosylation site but different peptide backbones, or even a group of N-glycans with a common structural property (such as all tri-antennary N-glycans) by taking the total count of the spectra identified as N-glycopeptides in the group.

A generic scoring function, i.e., the cosine similarity, was used to measure similarity between spectra in the clustering algorithm. More customized scoring functions, sometimes derived from machine learning methods, were shown to be advantageous of detecting similarities among CID or HCD MS/MS spectra of peptides (e.g., on signature ions resulting from backbone fragmentation). The cosine similarity is simple yet robust similarity measure to capture fragmentation patterns of complex MS/MS spectra.

There are several existing methods to control the false discovery rate in the references. For one example, randomized N-glycans as decoy database can be used, and in other examples precursor mass differences can be employed for false discovery rate control. The FDR is normally controlled by target-decoy-strategy in proteomics, with hypothesis that the chance that a scoring algorithm assigns spectra from both databases is equal. Thus, in proteomics studies, a 1:1 ratio of targets and decoys is commonly used; however, the same target-decoy approach is not well suited to evaluate the glycopeptide identification. When glycopeptide assignments are manually evaluated, research indicated that the assignment accuracy is significantly worse than what is predicted by the FDR. Some other studies have shown that using 1:1 target-decoy-based approaches in glycoproteomics can fail to evaluate false discovery rate. In the present disclosure, the samples are composed of both non-modified peptides and glycopeptides. To accurately evaluate the false discovery rate, the same spectral library searching procedure was applied in both sets of peptides (non-modified and glycopeptides). The false discovery rate for glycopeptides could be reflected by un-modified peptides, regardless the way of creating the decoy database and the size of the database.

The evaluation results showed that the concurrent approach can identify 105%-224% more spectra as glycopeptides compared to the conventional glycopeptide identification on individual datasets by using Byonic. The improvement of glycopeptide identification enabled the quantification of site-specific N-glycosylations using the simple but robust spectral counting approach that were commonly used for label-free protein quantification in proteomics, which lead to the discovery of potential biomarkers of protein glycosylations in liver cancer patients.

An example of focused analyses of N-linked glycopeptides in an immunopurified glycoprotein (Haptoglobin) is provided above. However, the spectral library searching approach can be extended to the general glycoproteomic studies of complex samples including both N-linked and O-linked glycopeptides, as long as multiple glycoproteomic datasets from related samples are available. In fact, the success of the presented approach relies on a comprehensive glycopeptide spectra library that can be used as the target of the search. Currently, the library is constructed from smaller experimental datasets. It will be useful to construct a curated glycopeptide spectral library by compiling the increasing amount of glycoproteomic data, in particular generated by using EThcD, which was demonstrated to contain more information for glycoproteomic analyses. Spectral library searching using such a comprehensive library will further improve glycopeptide identification from glycoproteomic data.

The present disclosure has described one or more preferred embodiments, and it should be appreciated that many equivalents, alternatives, variations, and modifications, aside from those expressly stated, are possible and within the scope of the invention.

We claim:

1. A method for identifying glycopeptides in mass spectrometry datasets, the method comprising:
accessing a plurality of mass spectrometry datasets, wherein each of the plurality of mass spectrometry datasets comprises mass spectrometry spectra acquired from a different sample;
generating spectral cluster data comprising a plurality of spectral clusters by clustering the plurality mass spectrometry spectra within each of the plurality of mass spectrometry datasets;
generating a consensus spectrum for each spectral cluster in the spectral cluster data;
generating a spectral library using the consensus spectra, wherein the spectral library comprises a plurality of labeled consensus spectra;
comparing the plurality of mass spectrometry spectra in the plurality of mass spectrometry datasets with the plurality of labeled consensus spectra within each of the plurality of related datasets; and
identifying the plurality of glycopeptides in the mass spectrometry spectra in the plurality of mass spectrometry datasets based on comparing the plurality of labeled consensus spectra with the mass spectrometry spectra in the plurality of mass spectrometry datasets.

2. The method of claim 1, wherein the spectral library is generated using the consensus spectra representing the clusters of spectra with charges +2 to +5 that are annotated as unmodified peptides or glycopeptides.

3. The method of claim 1, wherein generating the spectral library comprises:
performing a comparison of each of the consensus spectra to a glycopeptide database containing a plurality of spectra of a plurality of known glycopeptides;
identifying the plurality of glycopeptides in each of the plurality of consensus spectra based on the comparison; and
generating the plurality of labeled consensus spectra based on the plurality of glycopeptides identified in each of the plurality of consensus spectra.

4. The method of claim 3, wherein generating a plurality of labeled consensus spectra further includes selecting only one of the plurality of known glycopeptides that matches each of the plurality of consensus spectra with the highest frequency.

5. The method of claim 1, further comprising identifying a glycopeptide in each of the plurality of mass spectrometry spectra in the plurality of mass spectrometry datasets based on a cosine similarity that is greater than a threshold value between each of the plurality of mass spectrometry spectra in the plurality of mass spectrometry datasets and each of the plurality of labeled consensus spectra.

6. The method of claim 5, wherein the threshold value is between 0.6 and 0.8.

7. The method of claim 1, wherein generating spectral cluster data comprising a plurality of spectral clusters by clustering the plurality mass spectrometry spectra within each of the plurality of mass spectrometry datasets comprises a locality-sensitive hashing technique.

8. The method of claim 1, wherein identifying the plurality of glycopeptides in the mass spectrometry spectra in the plurality of mass spectrometry datasets based on comparing the plurality of labeled consensus spectra with the mass spectrometry spectra in the plurality of mass spectrometry datasets comprises a locality-sensitive hashing technique.

9. The method of claim 1, wherein the samples comprise blood serum.

10. The method of claim 1, wherein the samples comprise site-specific N-glycosylations in haptoglobin.

11. The method of claim 1, wherein the samples comprise N-linked and/or O-linked glycopeptides.

12. The method of claim 1, wherein the samples are immunopurified.

13. The method of claim 1, wherein the samples are digested and enriched.

14. A method for identifying glycopeptides in mass spectrometry datasets, the method comprising:
accessing a first plurality of mass spectrometry datasets, wherein each of the plurality of mass spectrometry datasets comprises mass spectrometry spectra acquired from a first multiplicity of samples;
generating spectral cluster data comprising a plurality of spectral clusters by clustering the plurality mass spectrometry spectra within each of the first plurality of mass spectrometry datasets;
generating a consensus spectrum for each spectral cluster in the spectral cluster data;
generating a spectral library using the consensus spectra, wherein the spectral library comprises a plurality of labeled consensus spectra;
comparing a second plurality of mass spectrometry spectra in a second plurality of mass spectrometry datasets generated from a second multiplicity of samples with the plurality of labeled consensus spectra; and
identifying the plurality of glycopeptides in the second multiplicity of samples based on comparing the plurality of labeled consensus spectra acquired from a first multiplicity of samples, with the mass spectrometry spectra in the second plurality of mass spectrometry datasets;
wherein the first multiplicity of samples and the second multiplicity of samples are prepared using the same experimental protocol.

15. The method of claim 14, wherein the first multiplicity of samples and the second multiplicity of samples comprise blood serum.

16. The method of claim 14, wherein the first multiplicity of samples and the second multiplicity of samples comprise site-specific N-glycosylations in haptoglobin.

17. The method of claim 14, wherein the first multiplicity of samples and the second multiplicity of samples comprise N-linked and/or O-linked glycopeptides.

18. The method of claim 14, wherein the first multiplicity of samples and the second multiplicity of samples are immunopurified.

19. The method of claim 14, wherein the first multiplicity of samples and the second multiplicity of samples are digested and enriched.

20. A system for identifying glycopeptides in multiple related datasets, the system comprising:
a processor configured to:
acquire a plurality of liquid chromatography coupled tandem mass spectrometry (LC-MS/MS) spectra from a plurality of related datasets;
cluster the plurality of LC-MS/MS spectra within each of the plurality of related datasets;
generate a spectral library using a consensus spectrum for each of the LC-MS/MS spectra within each of the plurality of related datasets;
perform a library search with a comparison of each of the consensus spectra to a glycopeptide database containing a plurality of spectra of a plurality of known glycopeptides;
identify the plurality of glycopeptides in each of the plurality of consensus spectra based on the comparison;
generate a plurality of labeled consensus spectra based on the plurality of glycopeptides identified in each of the plurality of consensus spectra;
compare the plurality of LC-MS/MS spectra with the plurality of labeled consensus spectra within each of the plurality of related datasets; and
identify the plurality of glycopeptides in the plurality of LC-MS/MS spectra based on the plurality of labeled consensus spectra.
